(19) United States
(12) Patent Application Publication
(10) Pub. No.: US 2024/0382700 A1
(43) Pub. Date: Nov. 21, 2024

(54) SPRAY HEAD AND DEVICE FOR INTRANASAL SPRAYING OF LIQUID MEDICINE

(71) Applicant: Nasal-Phyto (SZ) Pharmaceutical Technology Co., Ltd., Shenzhen, Guangdong (CN)

(72) Inventors: Jingyi Liu, Shanghai (CN); Zhutuo Chen, Shanghai (CN)

(21) Appl. No.: 18/268,398

(22) PCT Filed: May 12, 2023
(86) PCT No.: PCT/CN2023/093884

§ 371 Date: Jun. 20, 2023

(30) Foreign Application Priority Data
Jul. 28, 2022 (CN) 202221960466.2

(51) Int. Cl. A61M 11/00

(52) U.S. Cl. CPC A61M 11/006; A61M 2206/10; A61M 2210/0618

(57) ABSTRACT

The present disclosure provides a spray head and a device for intranasal spraying of liquid medicine, comprising: a nozzle, which has a flow channel running through the axial direction, and the distal and proximal ends of the flow channel are the flow channel outlet and inlet, respectively; a water core circumferentially arranged with a water trough configured inside the flow channel; and the outlet tube, curved in shape, comprising a parallel section and an inclined section integrally connected. The end of the inclined section extends from the flow channel inlet to the inside of the flow channel and is circumferentially sealed to the inner wall of the flow channel, so that when the fluid flows in from the parallel section, it flows out from the inclined section into the inside of the flow channel and after passing through the water trough, it ejects from flow channel outlet.

### TECHNICAL FIELD

The present disclosure relates to the technical field of medical equipment, in particular to a spray head and a device for intranasal spraying of liquid medicine.

### BACKGROUND

Blood-brain barrier (BBB) is the main obstacle for drugs to play a role in the treatment of central diseases. Due to the existence of BBB, the existing routes of drug delivery such as oral administration, intravenous injection, intramuscular injection, subcutaneous injection and other routes cannot effectively deliver drugs to the central nervous system (CNS). Because drugs cannot effectively cross the blood-brain barrier, they cannot play a role in the treatment of brain diseases. Moreover, as the dose of the drug administered increases, so do the systemic side effects.

Central routes of drug delivery include intracerebroventricular injection (ICV) and direct injection into the brain parenchyma and other methods. Although they are well-targeted and can deliver drugs directly to the injured brain region for therapeutic effect, they are invasive due to the craniotomy and may have postoperative complications such as infection. In addition, the existing central route of drug delivery is costly and financially burdensome for patients, making widespread application impractical for thousands of patients with brain disease. Therefore, finding a noninvasive central route of drug delivery that avoids obstruction of the BBB is crucial for the actual therapeutic effect of drugs in brain diseases.

Drug delivery through the nasal mucosa has been considered as a way of drug delivery that can be absorbed rapidly and efficiently. There are many microvilli on the nasal mucosa cells, which can greatly increase the effective area for drug absorption. There are abundant blood vessels and lymphatic vessels under the mucous membrane cells, and drugs can be directly absorbed into the systemic circulation through mucous membrane. In addition, the metabolic effect of enzymes in the nasal cavity is much smaller than that in the gastrointestinal tract, therefore, the nasal drug delivery system is receiving increasing attention.

The nasal spray device in the prior art cannot be precisely positioned to the olfactory region, resulting in reduced brain targeting. Conventional nasal spray drug is mainly deposited in the lower nasal tract, and the drug is absorbed across the membrane and then enters the bloodstream before crossing the BBB into the brain. However, the olfactory region is rich in olfactory nerves, which can bypass the BBB and enter the brain directly. In addition, since the drug enters the brain directly, precision and parallelism of drug delivery are particularly important.

In non-erect situations, patients holding the sprayers are prone to have differences each time, resulting in poor parallelism in the nasal deposition position of the drug solution, which may affect the efficacy. When patients treat themselves at home, it is difficult to be precise about the incoming nasal portion. How to ensure that the drug is sprayed to the olfactory region at the best angle becomes the key, therefore, a device with good targeting of high olfactory region, high accuracy of drug delivery, good parallelism, easy to use and carry, is needed to be designed to solve the shortcomings of the prior art.

### BRIEF SUMMARY

The purpose of the present disclosure is to provide a spray head and a device for intranasal spraying of liquid medicine, in view of defects in the prior art.

According to the disclosure, a spray head and a device for intranasal spraying of liquid medicine, comprising:
a nozzle, which has a flow channel running in an axial direction, and the distal and proximal ends of the flow channel are respectively connected with the outside through the flow channel outlet and inlet, the flow channel inlet is in the shape of a flared mouth.
a water core, being configured inside the flow channel and near the distal end of the flow channel circumferentially arranged with water troughs, the water core comprising water core frontend in a cylindrical structure and a water core backend, a plurality of said water troughs being evenly arranged along the circumference of the water core backend, the bottom surface of the water troughs being higher than or equal to the outer surface the water core frontend.
an outlet tube, which is set in a curved shape, comprises an integrally connected parallel section as well as an inclined section. Wherein the end of the inclined section extends from the flow channel inlet to the interior of the flow channel and against of the end of the water core backend, and is circumferentially sealed to the interior wall of the flow channel. When fluid flows from the parallel section, it flows from the inclined section into the interior of the flow channel and is ejected from the flow channel outlet after passing through water troughs.

The outlet tube and the nozzle are made of polypropylene or polyethylene, the outlet tube is prepared by the injection molding process.

Preferably, the proximal end of nozzle is configured with a limit structure so that when the distal end of the flow channel extends into the deepest position inside the nostril it can be positioned by the limit structure.

Preferably, the water trough extends in the direction of the water core axially.

Preferably, the outer diameter of the water core backend is larger than the outer diameter of the water core frontend.

Preferably, the end of the water core frontend is a circular plane, and the side of the flow channel outlet facing the water core frontend is configured with a cyclone slot.

Preferably, the flow channel accommodates the water core frontend towards the distal end is a step-like structure with gradually smaller space.

Preferably, the end of the water core backend is recessed and the recess is connected to the water trough.

Preferably, the flow channel has an inner concave bowl bottom concave structure at the distal end.

Preferably, the connection of the nozzle and the water core frontend is interference fit, snap or threaded connection.

A spray device for intranasal spraying of liquid medicine according to the present disclosure, comprising the spray head for intranasal spraying of liquid medicine, further comprising: spray nozzle, connected at the top to the parallel section for delivering the fluid; quantitative pump, connected at the top to the bottom of the spray nozzle to provide power for the delivery of the fluid; and reservoir for holding the fluid, the quantitative pump is attached to the reservoir and the bottom of the quantitative pump extending to the bottommost part of the interior of the reservoir.

Preferably, the top of the end of the spray nozzle connected to the parallel section of the outlet tube is provided in a concave configuration, the concave configuration may be of any shape, for example, it may be circular, fan-shaped, arrow-shaped, rectangular, square, trapezoidal, triangular, more preferably, arrow-shaped.

Preferably, the reservoir bottle is provided with another bottle sleeve outside adapted to the spray nozzle, the bottle sleeve may be provided in any shape, such as capsule type, round type, pentagonal type, hexagonal type, more preferably, capsule type.

Compared with the prior art, the present disclosure has the following beneficial effects.

The outlet tube in the present disclosure is set in a curved shape, which can keep the liquid part of the nasal spray upright, and then through the guiding effect of multiple water troughs set in the circumferential direction of the water core, it not only solves the problem of poor parallelism of the nasal deposition position of the drug, but also ensures that the drug is sprayed to the olfactory region at the best angle to achieve a high deposition rate in the olfactory region, thus improving the brain targeting of the drug, This improves drug targeting to the olfactory region, drug delivery accuracy, parallelism, patient compliance, and ease of use and portability.

The end of the front end of the water core in the present disclosure is a circular plane, and flow channel outlet towards the water core frontend is configured with a cyclone trough and a spray hole to form a coherent and integrated structure. Compared with the structure in which the cyclone trough is arranged on the water core and the spray hole are separated in the prior art, the impact weakened effect between the cyclone fluid and the spray hole wall is avoided. It makes the fluid rotation and the spray hole spraying at one go, and the spray effect is better.

The design of the nozzle limiting structure in the present disclosure can solve the defect that the length of the nasal part is difficult to precisely position when the patient administers the drug by himself and causes the drug effect to be reduced.

In the present disclosure, the water core is arranged into a plurality of water troughs, and the total water passing through volume is less than the water passing space formed by the water core frontend and the nozzle. It ensures that the spraying burst is moderate, which can ensure that the spraying is concentrated to the olfactory area, and also can keep the connecting parts tightly connected without detachment when spraying.

The disclosure can improve the deposition rate of drugs in the olfactory region and improve the therapeutic efficiency of drugs for neurological diseases.

By setting a concave structure just above the connection between the spray nozzle and the parallel section of the outlet tube, the plastic adhesive thickness of the spray nozzle sealing part is reduced, and the product will not produce shrinkage defects after injection molding, and the sealing is good, which solves the problem of liquid leakage due to poor sealing between the outlet tube and the spray nozzle in the field of nasal spray.

### BRIEF DESCRIPTION OF THE DRAWINGS

Other features, purposes, and advantages of the disclosure will become apparent by reading a detailed description of the non-restrictive embodiments with reference to the attached drawings below:

FIG. 1 shows a schematic diagram of the structural profile of the spray head in the present disclosure.

FIG. 2 shows a schematic diagram of the structure profile of the water core and the nozzle after assembly in the present disclosure.

FIG. 3 shows the structure diagram of the water core separated from the nozzle in the present disclosure.

FIG. 4 shows a schematic diagram of the structure of the device for intranasal spraying of liquid medicine in the present disclosure.

FIG. 5 shows a schematic diagram of the structural profile of the device for intranasal spraying of liquid medicine in the present disclosure.

FIG. 6 shows a schematic diagram of the structure explosion of the device for intranasal spraying of liquid medicine in the present disclosure.

FIG. 7 shows the structural diagram of the cross section of the cyclone trough.

FIG. 8 shows a schematic diagram of the structure explosion of the device for intranasal spraying of liquid medicine in the concave configuration of the spray nozzle in the present disclosure.

FIG. 9 shows a diagram of the device for intranasal spraying of liquid medicine in the concave configuration of the nozzle of the present disclosure.

Attached mark description:

| | |
|---|---|
| Outlet tube 1 | Flow channel 31 |
| Parallel section 11 | Flow Channel Outlet 311 |
| Inclined section 12 | Flow Channel Inlet 312 |
| Water core 2 | Limit structure 32 |
| Water Core Frontend 21 | Spray Nozzle 4 |
| Water core Backend 22 | Dust Cover 5 |
| Water Trough 221 | Quantitative Pump 6 |
| Nozzle 3 | Reservoir 7 |

### DETAILED DESCRIPTION

The present disclosure is described in detail below in connection with specific embodiments. The following embodiments will assist those technicians in the art to further understand the disclosure, but do not limit the disclosure in any way. It should be noted that, to ordinary technicians in the art, several variations and improvements can be made without departing from the present conception of the disclosure. These all belong to the scope of protection of the present disclosure.

### Embodiment 1

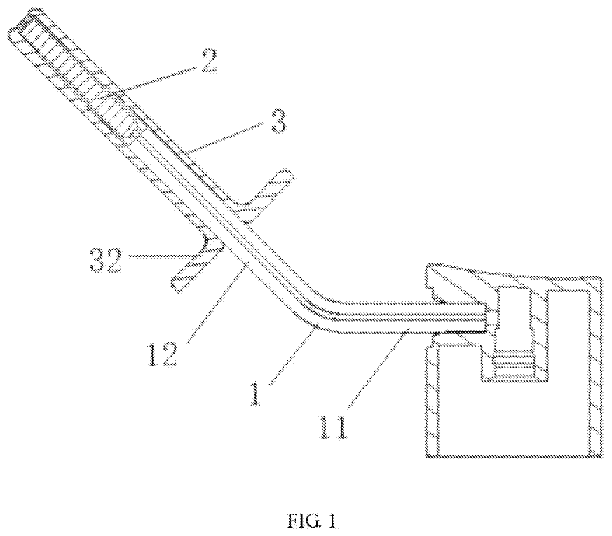

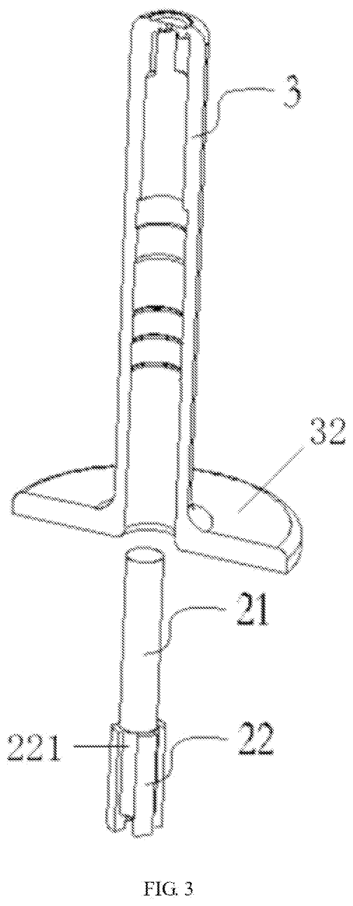

The present disclosure provides a spray head for intranasal spraying liquid medicine, including an outlet tube 1, a water core 2, and a nozzle 3, as shown in FIGS. 1, 2, and 3. The nozzle 3 has a flow channel 31 running in the axial direction, and the distal and proximal ends of the flow channel 31 are connected to the outside through the flow channel outlet 311 and the flow channel inlet 312, respectively. Water core 2 circumferentially arranged with the water trough 221, the water trough 221 preferably extended along the direction of the axial direction of the water core 2. The number of water troughs 221 can be set as one, or a plurality can be set uniformly or non-uniformly along the circumferential direction of the water core 2. The water trough 221 is configured inside the flow channel 31 and near the distal end of the flow channel 31. The outlet tube 1 is curved. The special curvature of the outlet tube 1 enhances patient compliance and facilitates upright pressing of the device during the injection of the liquid medicine, which is more conducive to the spraying of the liquid.

Moreover, when the patient uses it at home by himself, the limiting function of the nozzle 3 can guarantee the parallelism of the drug delivery, precise positioning and high targeting of the injection to the olfactory area and improve the deposition rate of the liquid in the olfactory area. The outlet tube 1 includes an integrally connected parallel section 11 and an inclined section 12. The parallel section 11 and the inclined section 12 are at a certain angle, and the end of the inclined section 12 extends from the flow channel inlet 312 to the inside of the flow channel 31. The end of the inclined section 12 is circumferentially sealed to the inner wall of the flow channel 31 so that when the fluid flows in from the parallel section 11, it flows out from the inclined section 12 into the interior of the flow channel 31 and is ejected from the flow channel outlet 311 after passing through the water troughs 221.

In practice, the outlet tube 1 and the nozzle 3 can be made of polypropylene or polyethylene, which can be chosen flexibly for specific applications. Preferably, the outlet tube 1 is prepared by the injection molding process.

It should be noted that, the flow channel 312 is flared to facilitate assembly at the end of the inclined section 12, the nozzle 3 is connected to water core frontend 21 so that the interior of the nozzle 3 forms a sealed cavity. Nozzle 3 and water core backend 22 can be connected by interference fit. snap or threaded connection, preferably interference fit.

Specifically, the proximal end of the nozzle 3 is configured with a limiting structure 32 such that the distal end of the flow channel 31 can be positioned by the limiting structure 32 when it extends to the deepest position inside the nostril. Thus, the patient can easily and conveniently make the distal end of the nozzle 3 reach the most suitable position for spraying inside the nostril by the limiting structure 32 during use, greatly improving the convenience and precision of the operation.

Further, the water core 2 includes a water core frontend 21 as well as a water core backend 22. The water troughs 221 is arranged at the water core backend 22, circumferentially arranged with a plurality of water troughs 221, the water core 2 comprises water core frontend 21 in a cylindrical structure and a water core backend 22, a plurality of said water troughs 221 being evenly arranged along the circumference of the water core backend 22, the bottom surface of the water troughs 221 being higher than or equal to the outer surface the water core frontend 21. The outer diameter of the water core backend 22 is preferably larger than the outer diameter of the water core frontend 21. And the total amount of water passing through the water space formed by the radial distance between water core backend 22 and the nozzle 3 is less than the total amount of water passing through the water trough 221.

In practice, the position of the flow channel 31 to accommodate water core frontend 21 towards the distal end is a step-like structure with gradually smaller space. The water core backend 22 has a concave end and is connected to the water trough 221. When the fluid flows here, the fluid first enters the inner concave and then enters the water trough 221, with a certain uniform diversion effect. The end of the water core frontend 21 is a circular plane. Flow channel outlet 311 towards the side of the water core frontend 21 is configured with cyclone trough. When the fluid flow to the cyclone trough to form a spray hole vortex chamber, and eventually presented in the formation of spray.

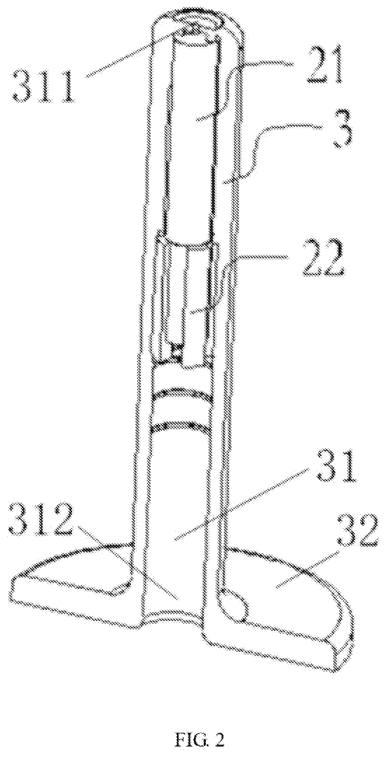

As shown in FIG. 2, the distal end of the flow channel 31 has an interior concave bowl bottom concave structure. The small pore size of the flow channel outlet 311 and the concave bowl bottom structure allow the ejected fluid to be dispersed and eventually ejected in a conical mist state.

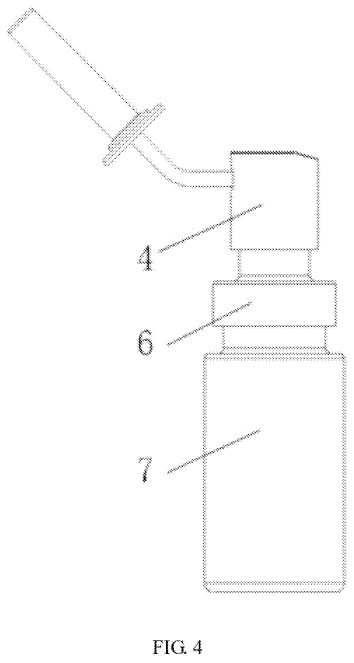

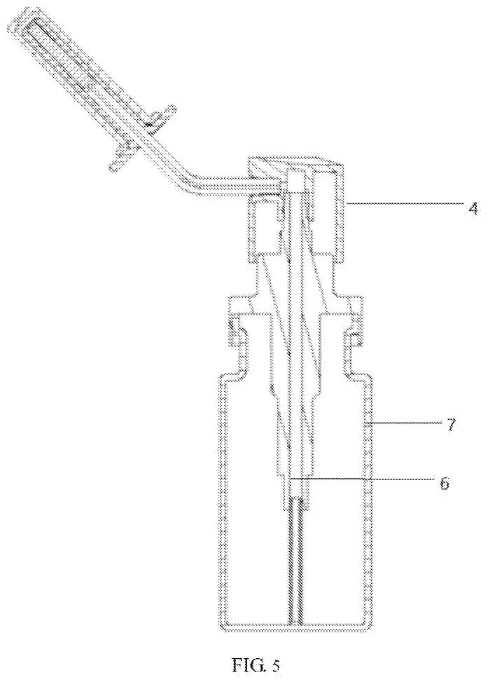

The present disclosure also provides a device for intranasal spraying liquid medicine, including a spray head for spraying liquid medicine into the nose, as shown in FIGS. 4 and 5, and also includes a spray nozzle 4, a quantitative pump 6, and reservoir 7. The top of the spray nozzle 4 is connected to a parallel section 11 for delivering fluid. The top of the quantitative pump 6 is connected to the bottom of the spray nozzle 4 to provide power for the delivery of fluid. The reservoir 7 is used to hold the fluid, and the quantitative pump 6 is fitted to the reservoir 7 and the bottom of the quantitative pump 6 extends to the bottommost part of the interior of the reservoir 7. The fluid in the present disclosure is liquid medicine, the liquid is sealed in the reservoir 7, and the end of the parallel section 11 of the outlet tube 1 is connected to the branch tube of the quantitative pump 6. Wherein the reservoir 7 can be selected from a pressure buckle bottle, a threaded bottle, etc.

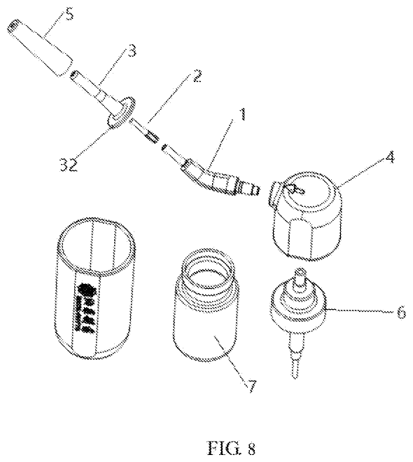

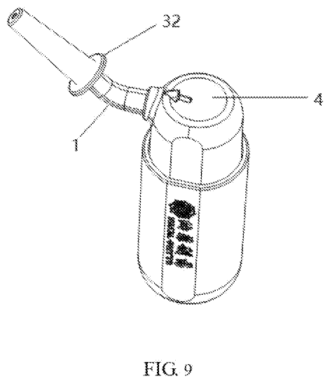

In a preferred embodiment, As shown in FIG. 8 and FIG. 9, the top of the end of the spray nozzle 4 connected to the parallel section of the outlet tube is provided in a concave configuration, the concave configuration may be of any shape, for example, it may be circular, fan-shaped, arrow-shaped, rectangular, square, trapezoidal, triangular, preferably, arrow-shaped.

In a preferred embodiment, the reservoir bottle is provided with another bottle sleeve outside adapted to the spray nozzle, the bottle sleeve may be provided in any shape, such as capsule type, round type, pentagonal type, hexagonal type, preferably, capsule type.

It should be noted that the quantitative pump 6 in the present disclosure adopts the prior art. which will not be repeated here.

### Embodiment 2

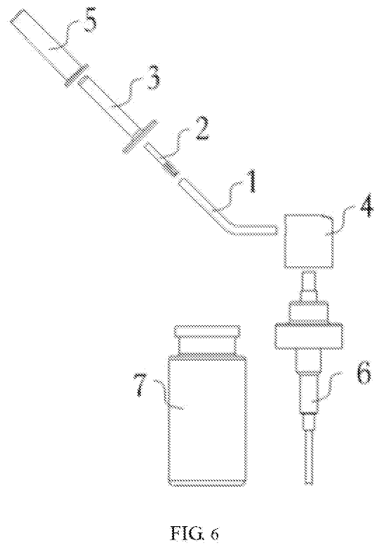

This embodiment provides a device for intranasal spraying liquid medicine, as shown in FIG. 6, including an outlet tube 1, a water core 2, and a nozzle 3. The outlet tube 1 is set in a curved shape with an angle, a parallel section 11 is connected to an external medicine source, and an inclined section 12 is connected to the nozzle 3, and the outlet end of the nozzle 3 is set with a spray hole, which is the flow channel outlet 311. The water core frontend 21 cooperates with the spray hole of the nozzle 3 to form a spray hole vortex chamber. The water core backend 22 of the nozzle 3 set up with the limit structure 32, which is integrally formed with the nozzle 3. The limit structure 32 in this embodiment is a circular structure. In the specific design, other structures can also be designed, such as fan ring, and multiple discontinuous fan ring structures. Specific design flexibility according to the shape of the aesthetic and practical needs, which will not be repeated here.

The water core 2 is a solid core of irregular cylindrical shape, which is a one-piece molding. The water core backend 22 set with one or more water troughs 221. Water core frontend 21 is connected with nozzle 3, and the outlet tube 1, the water core 2 and the nozzle 3 are fixed in axial position to each other.

It should be noted that the special curvature of the outlet tube 1 ensures that the liquid medicine is sprayed at an optimal angle to the olfactory area. When patients treat themselves at home, they will not have difficulty in precisely grasping the inlet portion. The precision and parallelism are the combined effect of the limiting structure 32 and the angle. High olfactory zone targeting, accuracy of drug delivery, parallelism, patient compliance, ease of use and portability are the advantages of the present disclosure.

In this embodiment, the water trough 221 is provided along the axial direction of the water core 2, and the water trough 221 may be rectangular, tangential circular, triangular, and other shapes.

In this embodiment, the difference between the internal radius of the nozzle 3 and the radius distance of the water core frontend 21 is 0.2 mm. The connection between the nozzle 3 and the outlet tube 1 is an interference connection.

In this embodiment, the angle of the bending shape of the outlet tube 1, i.e., the angle between the parallel section 11 and the inclined section 12, is greater than 90°, less than 180°; more preferably, it is 120° to 150°, for example, it can be 125°, 130°, 135°, 140°, 145°, etc.

In this embodiment, the length of the diameter of the limit structure 32 is greater than the diameter of the nostril.

In this embodiment, the distance from the flow channel outlet 311 of the nozzle 3 to the limiting structure 32 is 20 mm to 30 mm, more preferably, 22 mm to 28 mm, further preferably, 24 mm to 26 mm; most preferably, 25 mm. Within the acceptable comfort range of patients, the nasal cavity should be penetrated as far as possible to cooperate with the spray effect and improve the drug deposition rate in the olfactory area.

In this embodiment, the end surface of the flow channel outlet 311 of the nozzle 3 is provided with a curved surface to reduce the irritation of the nozzle 3 to the nasal cavity.

In this embodiment, the nozzle 3 is further provided with a removable dust cover 5 to prevent irritation to the nasal cavity caused by adhesive dust adhering to the flow channel outlet 311.

In this embodiment, the outlet tube 1, nozzle 3, and dust cover 5 are preferably made of polypropylene or polyethylene. In practical applications, the outlet tube 1, nozzle 3, and dust cover 5 can be other materials, to the extent that they meet the preservation of the medicine and facilitate the spraying of the medicine.

The principle of operation of the present disclosure for intranasal spraying of liquid medicine is as follows:

When the device is used, it is preferred to remove the dust cover 5. When using the product for the first time, press the Spray nozzle 4 with your finger and repeat the pressure X times until an uniform spray appears. Hold the body of the reservoir 7 in your hand, put your thumb on the spray nozzle 4, keep the bottle vertical when spraying, put the flow channel outlet 311 of the nozzle 3 facing your face and insert the nozzle 3 slowly into any nostril along the nasal septum until it touches the nozzle limit structure 32. At this point, press down the spray nozzle 4 once with the thumb to complete a spray drug delivery. Specifically, the spray can be repeated after a certain time interval, or the nozzle 3 can be removed and inserted into another nostril with the same action to complete the administration of another nostril.

The fluid flow process and the final formation of the spray in the present disclosure works as follows:

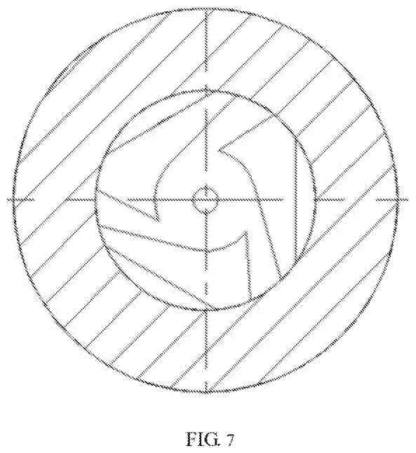

The end surface of water core frontend 21 is a circular plane, and the back side of the flow channel outlet 311 of the nozzle 3 is designed with a cyclone trough. Circular plane and the plane of the cyclone trough form a spray hole vortex chamber. The water trough 221 of the water core backend 22, the water core frontend 21 and the space formed between the nozzle 3 together to form the liquid jet channel. Liquid flow to the water core backend 22, through the jet channel, to reach the spray hole vortex chamber. When passing through the spray hole vortex chamber, the liquid passes through the channel of the cyclone trough (as shown in FIG. 7) to form several streams of liquid at the spray hole to form a mist, that is, to form tiny particles, thus forming a spray effect.

The present disclosure, in order to achieve the technical effect of direct injection of the medicinal solution into the nasal olfactory region, firstly controls the depth of entry into the nose. The distance from the spray hole of the nozzle 3 to the limiting structure 32 is preferably designed to be 25 mm, which can be precisely positioned to the olfactory region. Secondly, the outlet tube 1 is set to the most suitable angle for spraying liquid, which is conducive to keep the reservoir upright and facilitate the spraying operation and more conducive to deliver the liquid to the olfactory area. Finally, designed from the atomization morphology, the nozzle 3 is set with a cyclone trough on the flow channel outlet 311, which cooperates with the water core 2 to form a vortex chamber and deliver the liquid medicine directly to the olfactory region through the spray hole.

In the description of the application, it should be understood that the directions or positional relationships indicated by the terms “up”, “down”, “front”, “back”, “left”, “right”, “vertical”, “horizontal”, “top”, “bottom”, “inside”. “outside”, etc. are based on the directions or positional relationships shown in the drawings, and are only for the convenience of describing the application and simplifying the description, rather than indicating or implying that the device or element must have a specific orientation, be constructed and operate in a specific orientation, it cannot be understood as a limitation of the application.

Specific embodiments of the present disclosure have been described above. It is to be understood that the present disclosure is not limited to the particular embodiments described above, and that various variations or modifications may be made by those skilled in the art within the scope of the claims, which do not affect the substance of the present disclosure. The embodiments and features in the embodiments of the present application may be combined with each other at will, if there is no conflict.

1. A spray head for intranasal spraying of liquid medicine, characterized in that it comprises:
a nozzle (3) having a flow channel (31) running in an axial direction and distal and proximal ends of the flow channel (31) being connected to the outside through a flow channel outlet (311), and a flow channel inlet (312), respectively, and the flow channel inlet (312) is in the shape of a flared mouth,
a water core (2), being configured inside the flow channel (31) and near the distal end of the flow channel (31) circumferentially arranged with water troughs (221), the water core (2) comprising water core frontend (21) in a cylindrical structure and a water core backend (22), a plurality of the water troughs (221) being evenly arranged along the circumference of the water core backend (22), the bottom surface of the water troughs (221) being higher than or equal to an outer surface the water core frontend (21),
an outlet tube (1), which is set in a curved shape, comprises an integrally connected parallel section (11) as well as an inclined section (12), wherein the end of the inclined section (12) extends from the flow channel inlet (312) to the interior of the flow channel (31) and against the end of the water core backend (22), and is circumferentially sealed to the interior wall of the flow channel (31), when fluid flows from the parallel section (11), the fluid flows from the inclined section (12) into the interior of the flow channel (31) and is ejected from the flow channel outlet (311) after passing through the water troughs (221),
wherein the outlet tube (1) and nozzle (3) are made of polypropylene or polyethylene, and
wherein the outlet tube (1) is prepared by an injection molding process.

2. The spray head for intranasal spraying of liquid medicine according to claim 1, characterized in that the proximal end of the nozzle (3) is configured with a limit structure (32), when the distal end of the flow channel (31) extends into the deepest part of the interior of a nostril, the distal end of the flow channel (31) is positioned by the limit structure (32).

3. The spray head for intranasal spraying of liquid medicine according to claim 1, characterized in that the water troughs (221) extend in a direction of the water core (2) axially.

4. The spray head for intranasal spraying of liquid medicine according to claim 1, characterized in that an outer diameter of the water core backend (22) is larger than that of the water core frontend (21).

5. The spray head for intranasal spraying of liquid medicine according to claim 1, characterized in that the end of the water core frontend (21) is a circular plane, and the side of the flow channel outlet (311) facing the water core frontend (21) is configured with a cyclone slot.

6. The spray head for intranasal spraying of liquid medicine according to claim 1, characterized in that the flow channel (31) accommodates the water core frontend (21) towards the distal end is a step-like structure with gradually smaller space.

7. The spray head for intranasal spraying of liquid medicine according to claim 1, characterized in that the end of the water core backend (22) has a recess and the recess is connected to the the water troughs (221).

8. The spray head for intranasal spraying of liquid medicine according to claim 1, the flow channel (31) has an inner concave bowl bottom concave structure at the distal end.

9. The spray head for intranasal spraying of liquid medicine according to claim 1, characterized in that a connection of the nozzle (3) and the water core frontend (22) is interference fit, snap or threaded connection.

10. A device for intranasal spraying of liquid medicine characterized in that the device comprises the spray head for intranasal spraying of liquid medicine according to claim 1, the device further comprising:
spray nozzle (4), connected at the top to the parallel section (11) for delivering the fluid, quantitative pump (6), connected at the top to the bottom of the spray nozzle (4) to provide power for the delivery of the fluid, and
reservoir (7) for holding the fluid, wherein the quantitative pump (6) is attached to the reservoir (7) and the bottom of the quantitative pump (6) extending to the bottommost part of the interior of the reservoir (7).

11. The device for intranasal spraying of liquid medicine according to claim 10, characterized in that the top of the end of the spray nozzle (4) connected to the parallel section of the discharge tube is provided in a concave configuration, wherein the concave configuration is circular, fan-shaped, arrow-shaped, rectangular, square, trapezoidal, triangular, or arrow-shaped.

12. The device for intranasal spraying of liquid medicine according to claim 11, characterized in that, the reservoir bottle (7) is provided with another bottle sleeve outside adapted to the spray nozzle (4), the bottle sleeve is provided as capsule type, round type, pentagonal type, hexagonal type, or capsule type.
